(19) United States
(12) Patent Application Publication
(10) Pub. No.: US 2008/0249412 A1
(43) Pub. Date: Oct. 9, 2008

(54) Preoperative and Intra-Operative Lens Hardness Measurement by Ultrasound

(75) Inventors: Chih-Chung Huang, Los Angeles, CA (US); K. Kirk Shung, Monterey Park, CA (US); Mark S. Humayun, Glendale, CA (US); Hossein Ameri, Alhambra, CA (US)

Correspondence Address: MCDERMOTT WILL & EMERY LLP, 28 STATE STREET, BOSTON, MA, 02109-1775, US

(73) Assignee: DOHENY EYE INSTITUTE, Los Angeles, CA (US)

(21) Appl. No.: 12/061,147

(22) Filed: Apr. 2, 2008

Related U.S. Application Data
(60) Provisional application No. 60/909,496, filed on Apr. 2, 2007.
(60) Provisional application No. 60/909,522, filed on Apr. 2, 2007.
(60) Provisional application No. 60/911,385, filed on Apr. 12, 2007.
(60) Provisional application No. 61/030,075, filed on Feb. 20, 2008.

(51) Int. Cl. A61B 8/10

(52) U.S. Cl. 600/442

(57) ABSTRACT

The present disclosure provides methods, systems, techniques and apparatus related to an instrumentation used to classify tissue before and/or during removal. The tissue can include any hard tissue/soft tissue interface, such as a cataract within a lens. Before an operation, an ultrasound image can be provided that gives a full scan of the targeted region, e.g., a lens, and provide a hardness profile. The profile could include a two or three dimensional map of the tissue hardness, assisting a surgeon to choose a suitable surgical procedure and strategy. During the operation, hardness measurements can be carried out real-time, in a constant manner, while the surgeon is working. This data can allow the handpiece to automatically adjust to the surgical conditions including the tissue hardness, increasing surgical performance, decreasing surgical procedure time and reducing the rate of complications.

### RELATED APPLICATION

This application claims the benefit of U.S. Provisional Patent Application No. 60/909,496 filed 2 Apr. 2007, the entire content of which is incorporated herein by reference. This application is also related to U.S. Provisional Patent Application No. 60/909,522 filed 2 Apr. 2007 and U.S. patent application Ser. No. ______ and entitled “Thrombolysis in Retinal Vessels with Ultrasound” filed 2 Apr. 2008; and also U.S. Provisional Patent Applications No. 60/911,385 filed 12 Apr. 2007, and No. 61/030,075 filed 20 Feb. 2008, the entire contents of all of which applications are incorporated herein by reference.

### BACKGROUND

A cataract is an opacity that develops in the crystalline lens of the eye or in its envelope. Cataracts decrease visual acuity and eventually result in blindness of the associate eye. Some cataracts appear in infancy or in childhood, but most develop in older individuals. Early on in the development of age-related cataract the power of the crystalline lens may be increased, causing near-sightedness (myopia), and the gradual yellowing and opacification of the lens may reduce the perception of blue colors. Cataracts typically progress slowly to cause vision loss and are potentially blinding if untreated. When left untreated cataracts can cause phacomorphic glaucoma. Very advanced cataracts with weak zonules are liable to dislocation anteriorly or posteriorly.

Cataracts are typically classified according to their color and pattern during eye examination with slit lamp. This is done with a five-scale grading approach. This technique is very subjective and can be inaccurate, and surgeons may find that what appeared to be a soft cataract is actually very hard. Cataract surgery is a delicate procedure and a surgeon's experience plays a significant role in the outcome. Unfortunately the inexperienced surgeons are more likely to misjudge the hardness of the lens, and this also increases the rate of corneal endothelial cell damage and other more serious complications.

Prior art surgical techniques have commonly employed a handpiece/probe (“phaco probe”) for the phacoemulsification of cataract tissue. Prior art phaco probes do not adjust the ultrasound power setting automatically. As a result, the surgeon has to select an ultrasound power setting at the beginning of the surgery and rely on his/her surgical technique to properly remove the cataract. Although, the surgeon can change the setting at any time during the surgery, the newly adopted setting depends on the surgeon's judgment. Improper use of ultrasound power increases the rate of complications.

Common cataract probes use an ultrasound drive frequency to emulsify lens. The tip either oscillates in a longitudinal or torsional motion to cut and remove lens. There are also commercial probes which use other technologies such as laser but currently they are used for soft lenses and by far ultrasound probes are the most effective.

### SUMMARY OF THE DISCLOSURE

The present disclosure provides methods, systems, techniques and apparatus providing for classification and/or tissue characterization of hard-soft tissue interfaces. Exemplary embodiments can be utilized for classification of a cataract lens before and/or during removal. Embodiments of the present disclosure can utilize and work with ultrasonic transducers or needle probes having piezoelectric material.

Ultrasound imaging techniques according to the present disclosure can be used prior to an operation to give a full scan of hard-soft tissue interfaces, e.g., the lens and a cataract, and an ultrasound image providing a hardness profile. The profile could include a two or three dimensional map of the targeted tissue/region, e.g., lens hardness. This can assist the surgeon to choose a suitable surgical procedure and strategy.

In further embodiments, ultrasound imaging techniques according to the present disclosure can be used during an operation, with hardness measurements being carried out in real-time, in a constant manner, while the surgeon is working. Data provided by such measurements can allow a handpiece to automatically adjust to the surgical conditions, including e.g., including the (lens) hardness, increasing surgical performance, decreasing surgical procedure time and reducing the rate of complications.

Further embodiments of the present disclosure can includes systems for performing ultrasound imaging techniques described herein. Such systems can include an ultrasonic probe, with control electronics for controlling ultrasonic energy emitted from the probe as well as reception/detection electronics for receiving energy reflected back to the probe and processing/displaying information from the reflected energy, e.g., as a hardness profile. The systems can, in exemplary embodiments, include an ultrasonic probe coupled or used with a phaco tip, an ultrasound pulser/receiver, and a digital signal processor (“DSP”). Such systems can further include a handpiece and associated microsurgical system.

Other features and advantages of the present disclosure will be understood upon reading and understanding the detailed description of exemplary embodiments, described herein, in conjunction with reference to the drawings.

### BRIEF DESCRIPTION OF THE DRAWINGS

Aspects of the disclosure may be more fully understood from the following description when read together with the accompanying drawings, which are to be regarded as illustrative in nature, and not as limiting. The drawings are not necessarily to scale, emphasis instead being placed on the principles of the disclosure. In the drawings:

FIG. 1 depicts a diagrammatic view of a configuration with an ultrasonic probe for measuring the lens hardness in a small area, according to an embodiment of the present disclosure;

FIG. 2A and FIG. 2B depict profiles of the sound velocity ultrasound attenuation coefficient, respectively, in a nucleus of a cataract lens;

FIG. 3 depicts a perspective view with cutout of an exemplary 3D image of the hardness of a lens, showing a hard nucleus in the center;

FIG. 4 depicts a block of ultrasound system according to an embodiment of the present disclosure;

FIG. 5 depicts a cross section view of an exemplary embodiment of a needle transducer in accordance with the present disclosure;

FIG. 6 depicts an embodiment of a needle transducer and associated reflector; and

FIG. 7 depicts a method of measuring lens hardness with ultrasound according to exemplary embodiments of the present disclosure.

While certain figures are shown, one skilled in the art will appreciate that the embodiments depicted in the drawings are illustrative and that variations of those shown, as well as other embodiments described herein, may be envisioned and practiced within the scope of the present disclosure.

### DETAILED DESCRIPTION

The present disclosure provides methods, systems, techniques and apparatus providing for classification and/or tissue characterization of hard-soft tissue interfaces. Exemplary embodiments can be utilized for classification of a cataract lens before and/or during removal. Embodiments of the present disclosure can utilize and work with ultrasonic transducers or needle probes having piezoelectric material. Exemplary embodiments of the present disclosure can utilize and work with either longitudinal or torsional motion of an associated phaco probe.

Preoperative embodiments of the present disclosure can be used before a desired surgery, e.g., cataract operations/procedures. For such embodiments, an ultrasound image can give a full scan of the targeted tissue/region, e.g., lens, and provide a hardness profile. The profile may include a two or three dimensional map of the tissue/region, e.g., lens, hardness. Such profiles can assist a surgeon in choosing a suitable surgical procedure and strategy.

Intra-operative embodiments of the present disclosure can be used during a desired surgery, e.g., cataract operations/procedures. For such embodiments, lens hardness measurements can be carried out in real-time, in a constant or sequential manner, and while the surgeon is working. The data produced can allow a surgical handpiece, which can include an ultrasonic probe, to automatically adjust to the surgical conditions, e.g., the lens hardness, to thereby increasing surgical performance, and decreasing surgical complications.

For embodiments according to the present disclosure, various physical parameters (e.g., sound velocity, attenuation, and direct elastic modulus measurements) of a hard tissue/soft tissue interface (e.g., a cataract within a lens) can be correlated to mechanical measurements, such as hardness. Also, correlation between the ultrasound measurements and total operating energy during surgery (e.g., operating power×time) can correspondingly be made. The regional change of acoustic parameters corresponding to the variation of hardness in the tissue/region, e.g., cataract lens, can be estimated (e.g., before the phacoemulsification surgical operation). Such correlations and estimation, as described below, have been supported and verified by experiments by the present inventors on enucleated porcine eyes, which showed that ultrasound velocity and attenuation can be used to measure the focal points of lens hardness in the center or periphery of the lens.

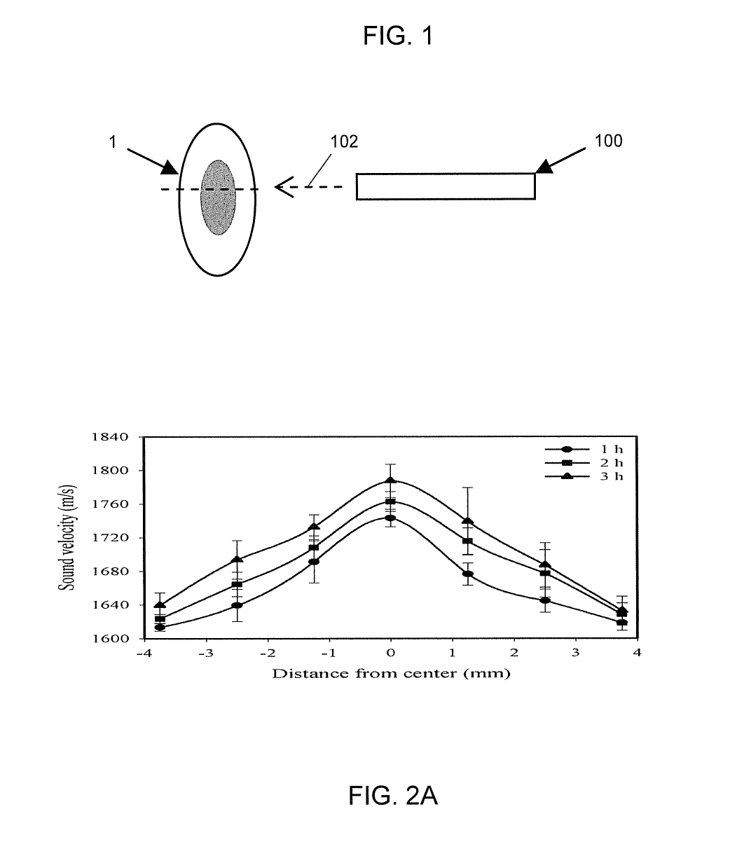

By pointing or directing an ultrasound transducer to a given area of a patient, e.g., a lens, it is possible to measure the tissue (e.g., lens) hardness in multiple points throughout the tissue thickness using sound velocity or attenuation coefficient. FIG. 1 depicts a diagrammatic view of a configuration with an ultrasonic probe for measuring lens hardness in a small area, according to an embodiment of the present disclosure. In FIG. 1, ultrasonic probe 100 is shown directing ultrasonic energy 102 to an eye lens 1. While an eye lens is depicted, the ultrasonic technique may be used for any other hard tissue interface (or gradation) with softer tissue in the body to help with excision of the softer or harder tissue.

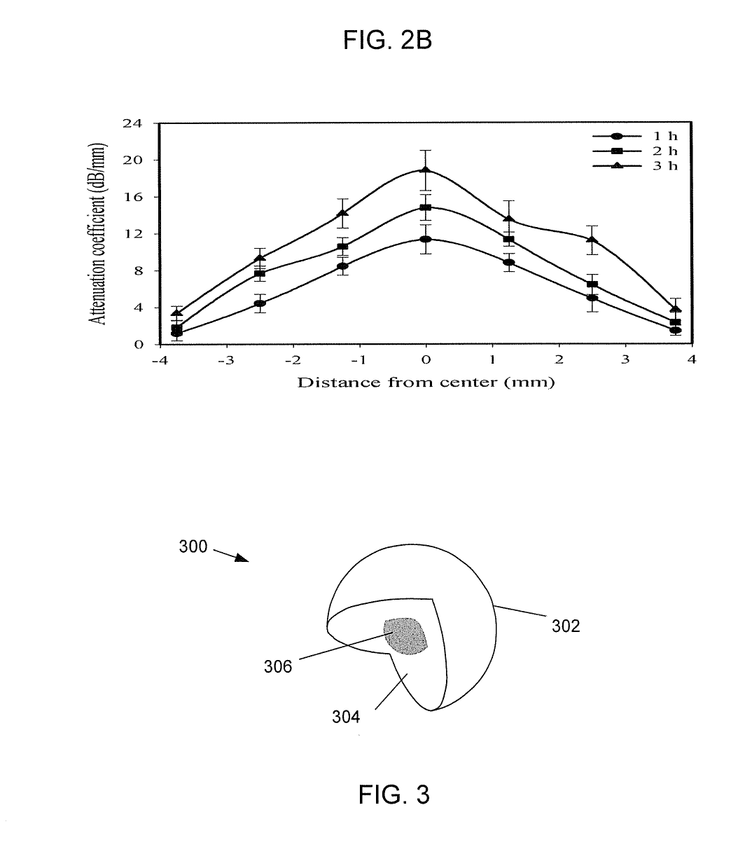

FIG. 2A and FIG. 2B depict profiles of the sound velocity ultrasound attenuation coefficient, respectively, in a nucleus of a cataract lens. For exemplary embodiments. As can be seen in FIGS. 2A and 2B, the sound velocity and attenuation are both maximum in the center of the lens, which is the hardest part.

By combining multiple small transducers, or using a larger transducer, it is possible to measure the lens hardness in a large area. A computer can combine the information and generate a 3D image of the lens hardness. FIG. 3 depicts a perspective view with cutout of an exemplary 3D image 300 of the hardness of a lens 302, showing a less dense region 304 surrounding a denser, hard nucleus 306 in the center.

With continued reference to FIG. 3, an image of a lens may be color coded; i.e. based on the hardness of a given area different colors are displayed in the image, to make the image more user friendly. Alternatively, the image could be on gray scale. The measurements can be taken while the patient is lying down, sitting up or standing up. The measurements can be taken through the closed eyelids or following instillation of topical anesthetic medications and after applying gel or other medium on the cornea.

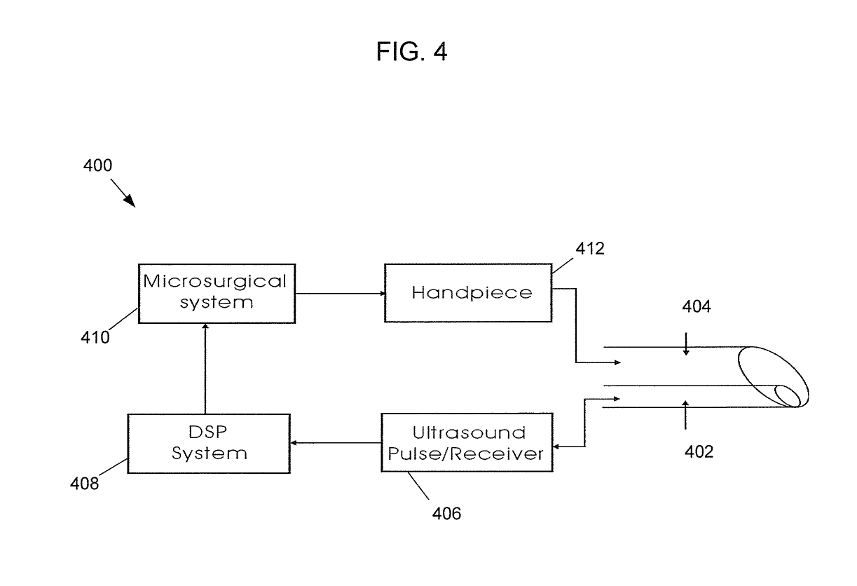

For measurements described herein, an ultrasound probe may be hand held or it may be fixed on a stand or other equipments, or it may be an integral part of the measuring device. In order to measure the lens hardness during the operation, a high frequency ultrasound needle transducer with a small size can be set outside/inside on the phaco tip. This design can allow a surgeon to measure the local hardness of cataract lens during the phacoemulsification surgical operation. FIG. 4 depicts a block of ultrasound system 400 according to an exemplary embodiment of the present disclosure;

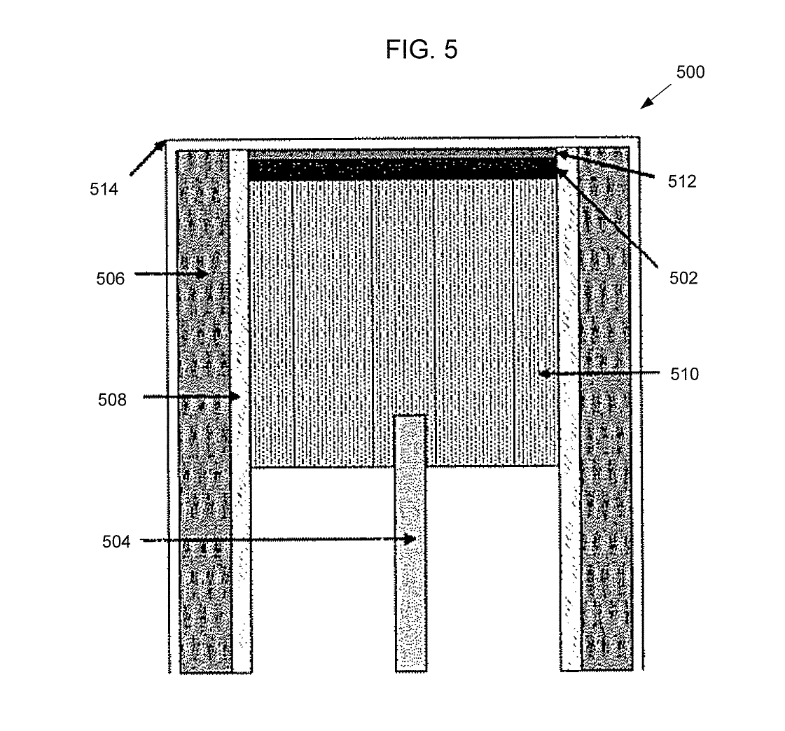

With reference to FIG. 4, system 400 can include a pulser/receiver 406 system with a suitable bandwidth, e.g., a 200-MHz bandwidth. Pulser/receiver 406 can be used to drive a needle transducer 402 for transmitting and receiving ultrasound signals with different pulse repetition frequency. The needle transducer can include a reflector (e.g., as shown in FIG. 5) and can be part of a phaco tip 404. The phaco top 404 can be part of or implemented with a handpiece 412 that is part of a microsurgical system 410.

Continuing with the description of FIG. 4, radio-frequency signals received from the reflector can be amplified and filtered using a built-in variable-gain amplifier (e.g., a 54 dB variable-gain amplifier) and a band-pass filter within or connected to the pulser/receiver. The RF signals can be passed through an electronic limiter for protection purposes and subsequently sampled at a sampling frequency, e.g., of 200 MHz, by a digital signal processing system (“DSP”) system 408.

The average RF signals from A-line signals can be acquired and stored in the DSP system 408, and then the signals are calculated to assess the local hardness of cataract lenses. For such hardness determination, the pulse-echo approach is preferably used to measure sound velocity in lens. This method is based on measurement of the time of flight of the ultrasonic pulses reflected from the reflector. For such time of flight techniques, the sound velocity V can be given by:

$$V = \frac{2 d}{t} ; \qquad ( 1 )$$

where d is the distance between transducer and reflector and t is the time of flight.

The ultrasonic frequency-dependent attenuation coefficient can be calculated using the following equation:

$$α ( f ) = \frac{20}{2 d} ( {log}_{10} \frac{{A}_{1} ( f )}{{A}_{2} ( f )} ) ; \qquad ( 2 )$$

where d is the distance between transducer and reflector, and A₁ (f) and A₂ (f) are the amplitude spectra computed from echoes received from the reflector in water and the reflector with lens tissue interposed, respectively, e.g., using a 1024-point fast Fourier transform. The regional variations of acoustic properties corresponding to the hardness in different kinds of tissue (e.g., cataracts) can be detected by needle transducer, e.g., transducer 402. Microsurgical system 410 can change the setup of phaco energy (e.g., including the power level and duty cycle) according to the indication from DSP system 408. At the same time, the handpiece 412 can be controlled automatically by the microsurgical system 410 to change the power level of the phaco tip 404. In exemplary embodiments, the whole process time can be less than 0.1 ms.

FIG. 5 depicts a cross-section view of an exemplary embodiment of a suitable ultrasonic needle transducer 500 in accordance with the present disclosure. As shown in FIG. 5, the probe 500 can include a piezoelectric material 502 disposed with a needle housing 106. The piezoelectric material 502 can be any suitable active piezoelectric material. One suitable piezoelectric material is lead magnesium niobate lead titanate (e.g., PNM-33% PT). The piezoelectric material may be attached (directly or indirectly, and with suitable electrical configuration/connection) to an electrical connector 504 by suitable fabrication/construction techniques. For example, Cr/Au electrodes can be used to connect the piezoelectric material 502 to the electrical connector 504, though other conductive material(s) may be used. Housing 506 can be of a desired diameter and material, e.g., steel of 1 mm diameter, which size can be suitable (or selected) for insertion into an ocular incision. The needle housing 506 can surround a tube 508 of electrically insulating/isolating material, e.g., made of polyimide fabricated by suitable techniques. The electrical connector may be one suitable for connection to a control system configured to control the production of acoustic energy from the transducer.

Continuing with the description of probe 500, a conductive backing material 510 can be located between the piezoelectric material 502 and the electrical connector 504. A matching layer 512 may be located on or adjacent to the side of the probe from which acoustic energy is to be produced. A protective coating 514 may optionally be present, with parylene being an exemplary material for the protective coating, though others may be used.

Needle probes according to the present disclosure, e.g., a lens hardness measurement probe, can be combined with various endoscopes used throughout body cavities to evaluate tumors such as melanoma, etc. Such probes can also be combined to or implemented with cryogenic (cryo), laser, illumination, and/or cautery probes used for various parts of body, including internal body cavities

To facilitate placement of an ultrasonic transducer, e.g., transducer 500, on the side of the phaco tip, e.g., phaco tip 404 of FIG. 4, the size of the transducer can be tailored as desired, e.g., to preferably smaller than 1 mm. In exemplary embodiments, a PMN-PT single crystal can be suitable for this application. For example, a 50 μm thick (001) poled PMN-PT single crystal with a size of 0.4 mm by 0.4 mm may be used as the active material of the transducer due to good electromechanical coupling coefficient afforded by such a crystal, high piezoelectric constant (d₃₃) and lower dielectric loss. The matching layer can be made by Insulcast 501 and Insulcure 9 and silver particles, in exemplary embodiments. The E-solder 3022 can be used as a conductive backing material. The single crystal can be housed using Epotek 301 within a polyimide tube with a small diameter. An electrical connector SMA-174 can be fixed to the conductive backing using the conductive epoxy. The active element within a polyimide tube can be housed in a needle; the polyimide tube provided electrical isolation from the needle. A vapor-deposited parylene layer with a thickness of 14 μm can be used to coat the aperture and the needle housing, for exemplary embodiments.

The angle of needle tip can be built at a desired angle (e.g., at a range from 30 to 60° from the longitudinal axis of the needle probe). Also, the length of needle probe transducer and the range of the outer diameter of the transducer can be constructed/selected as desired. For example, the length of the needle probe can be up to 45 mm and the he range of the outer diameter of transducer can be from 0.5 to 0.9 mm, in exemplary embodiments.

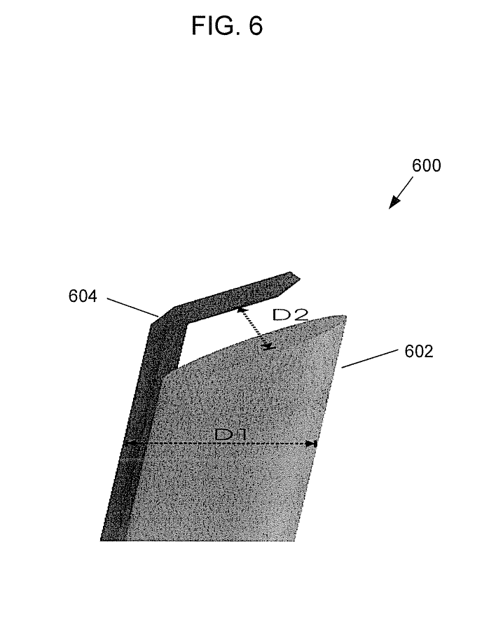

As shown in FIG. 6, to facilitate measurement of the sound velocity and ultrasound attenuation in a lens, a small metal reflector can be bound on the side of needle transducer as a reference target. As shown in FIG. 6, a needle transducer 602 can be used with reflector 604 for system 600.

The diameter D1 of the needle probe 602 can be selected/designed as desired. In exemplary embodiments, D1 can range from about 0.5 mm to about 0.9 mm. The distance D2 between transducer 602 and reflector 604 can be made/selected as desired. In exemplary embodiments, the distance D2 can be from about 0.2 to about 0.7 mm. The center frequency of needle transducer can be selected as desired, e.g., approximately 44-46 MHz, e.g., as for the embodiment depicted in FIG. 6.

The high frequency needle transducer can be placed inside or outside on the phaco tip, e.g., a transducer may be placed in the handle of the phaco hand piece. A needle transducer may be separate from the phaco hand piece and may be introduced into the eye from another port, in alternate embodiments

Accordingly, the present disclosure can provide one or more advantages compared to previous techniques. Full scan of the lens can provide a detailed and accurate measurement of lens hardness before the surgery. It is an objective method, and one that gives improved data before the procedure and allows selection of the appropriate surgical method, probes and settings. This can be used to characterize current cataract research/studies. Surgical outcomes can be improved by reducing the rate of complications.

Real-time feedback during the surgery can be provided with fewer surgical complications. Faster removal (more cases per surgeon per year) can also be realized. Embodiments of the present disclosure can be used to improves fluidic stability within the eye, and may be operable to automatically adjust according to the lens hardness and specific procedure circumstances.

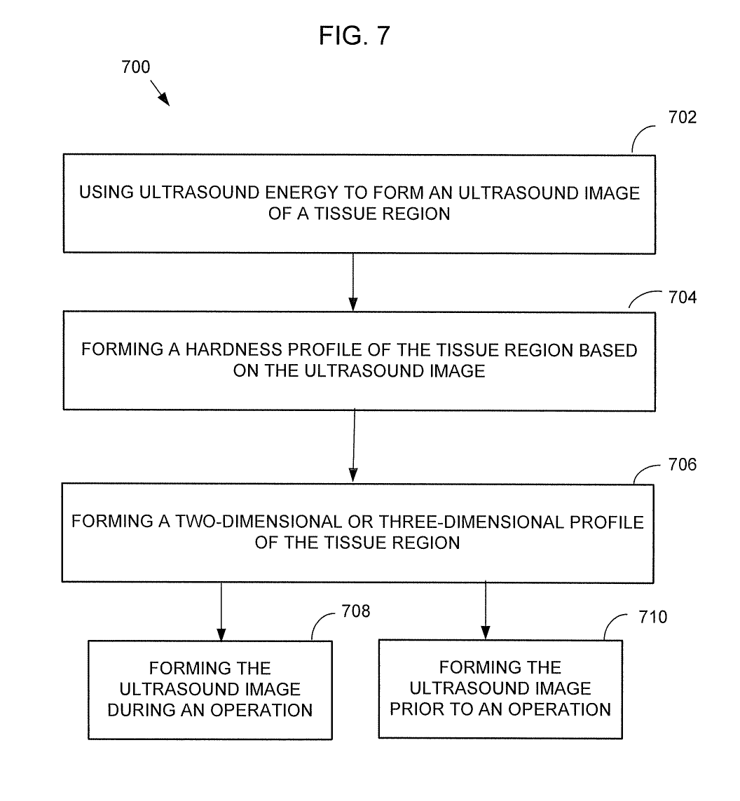

FIG. 7 depicts a method 700 of classifying a tissue region according to an embodiment of the present disclosure. The targeted tissue region can include any hard tissue/soft tissue interface. Exemplary embodiments can be used for classification of a cataract within an eye/lens. Ultrasound energy may be used to form an ultrasound image of a targeted tissue region (e.g., cataract lens), as described at 702. A hardness profile of the targeted region can be formed based on the ultrasound image. A two-dimensional or three-dimensional profile of the targeted region can be formed, as described at 706.

Continuing with the description of FIG. 7, an ultrasound image of a targeted region can be formed during operation on the region, as described at 708. Also, or alternatively, an ultrasound image can be formed prior to an operation on the region, as described at 710.

### Experimental Verification

Exemplary embodiments of lens classification using ultrasound techniques according to the present disclosure have been tested and found to be satisfactory a series of experiments using porcine eyes. For such tested embodiments, a 46 MHz high frequency needle transducer (similar to that described for FIG. 5) with a diameter of 0.5 mm was designed and fabricated for intraoperative measurement of acoustic properties in lens.

Multiple techniques have thus been proven (e.g., sound velocity, attenuation, and direct elastic modulus measurements) and correlated to mechanical measurements. Surgical correlation between the ultrasound measurements and total operating energy (operating power×time) have correspondingly been made. The regional change of acoustic parameters corresponding to the variation of hardness in cataract lens can be estimated before the phacoemulsification surgical operation. This is supported by the experiments on enucleated porcine eyes which showed that ultrasound velocity and attenuation could be used to measure the focal points of lens hardness in the center or periphery of the lens.

Accordingly, embodiments of the present disclosure can provide for measurement of lens hardness by pointing a ultrasound transducer to a given area of the lens. Such measurements can be taken or used for multiple points throughout the lens thickness using sound velocity or attenuation coefficient.

In exemplary embodiments, providing a hardness profile comprises forming a hardness profile in real time. A hardness profile can be formed multiple times during an operation. An ultrasonic hand piece used for the surgery can be adjusted during surgery based on lens hardness measurements. The hardness profile can be based on sound velocity or an ultrasonic frequency-dependent attenuation coefficient.

While certain embodiments have been described herein, it will be understood by one skilled in the art that the methods, systems, and apparatus of the present disclosure may be embodied in other specific forms without departing from the spirit thereof. For example, while certain piezoelectric materials have been mentioned specifically, others may be used within the scope of the present disclosure.

Additionally, ultrasonic transducers or needle probes according to the present disclosure, e.g., a lens hardness measurement probe, can be combined with various endoscopes used throughout body cavities to evaluate tumors such as melanoma, etc. Such probes can also be combined to or implemented with cryogenic (cryo), laser, illumination, and/or cautery probes used for various parts of body, including internal body cavities

Accordingly, the embodiments described herein are to be considered in all respects as illustrative of the present disclosure and not restrictive.

What is claimed is:

1. A method of classifying a tissue region, the method comprising:
using ultrasound energy to form an ultrasound image of the tissue region; and
forming a hardness profile of the tissue region based on the ultrasound image.

2. The method of claim 1, wherein the ultrasound image is formed prior to an operation on the tissue region.

3. The method of claim 1, wherein the ultrasound image of a cataract lens is formed during an operation on the tissue region.

4. The method of claim 1, further comprising forming a two-dimensional profile of the tissue region.

5. The method of claim 1, further comprising forming a three-dimensional profile of the tissue region.

6. The method of claim 3, wherein providing a hardness profile comprises forming a hardness profile in real time.

7. The method of claim 6, wherein a hardness profile is formed one or more times during an operation.

8. The method of claim 7, wherein the hardness profile is formed constantly during an operation.

9. The method of claim 7, wherein an ultrasonic hand piece used for the surgery is adjusted during surgery based on lens hardness measurements.

10. The method of claim 9, wherein an adjustment is made manually.

11. The method of claim 9, wherein an adjustment is made automatically.

12. The method of claim 1, wherein the hardness profile is based on sound velocity or an ultrasonic frequency-dependent attenuation coefficient.

13. The method of claim 1, wherein using ultrasonic energy comprises using an ultrasonic output at about 1 MHz to about 50 MHz.

14. The method of claim 13, wherein using ultrasonic energy comprises using an ultrasonic output at about 44 MHz to about 46 MHz.

15. The method of claim 14, wherein the output is about 45 MHz.

16. The method of claim 1, wherein the tissue region comprises a cataract.

17. An ultrasonic system for tissue hardness measurement, the system comprising:
an ultrasonic probe configured and arranged to produce an ultrasonic output;
control means operatively coupled to the probe for controlling operation of the probe; and
means for forming a hardness profile of a tissue region.

18. The system of claim 17, wherein the measurement means is configured and arranged to form the hardness profile based on sound velocity or an ultrasonic frequency-dependent attenuation coefficient.

19. The system of claim 17, wherein the ultrasonic probe comprises a phaco tip.

20. The system of claim 17, wherein means for forming a hardness profile is configured and arranged to form the hardness profile based on sound velocity or an ultrasonic frequency-dependent attenuation coefficient.

21. The system of claim 17, wherein the ultrasonic probe comprises a PMN-PT single crystal material.

22. The system of claim 21, wherein the ultrasonic probe is configured and arranged to produce an ultrasonic output at about 40 MHz to about 50 MHz.

23. The system of claim 22, wherein the output is about 44 MHz to about 46 MHz.

24. The system of claim 23, wherein the output is about 45 MHz.

25. The system of claim 17, wherein the probe is configured and arranged for insertion into an eye.
